(19) United States
(12) Patent Application Publication
(10) Pub. No.: US 2026/0199091 A1
(43) Pub. Date: Jul. 16, 2026

(54) DETACHABLE ANNULOPLASTY RING HOLDER

(71) Applicant: Edwards Lifesciences Corporation, Irvine, CA (US)

(72) Inventors: Hilda Z. Fann, Santa Ana, CA (US); David S. Lin, Irvine, CA (US)

(21) Appl. No.: 19/560,515

(22) Filed: Mar. 9, 2026

Related U.S. Application Data
(63) Continuation of application No. PCT/US2024/046565, filed on Sep. 13, 2024.
(60) Provisional application No. 63/582,809, filed on Sep. 14, 2023.

(51) Int. Cl. A61F 2/24

(52) U.S. Cl. CPC A61F 2/2466; A61F 2/2448; A61F 2220/0075; A61F 2250/001

(57) ABSTRACT

A holder for an annuloplasty ring that detaches with one cut. The annuloplasty ring mounts around a periphery of the holder without the use of connecting sutures. The holder has a proximal plate defining a lip on one side of the ring, and a distal retainer that defines a second lip on the other side, the two lips retaining the ring around the holder periphery. The distal retainer is formed by two hinged wings. A truss suture maintains the holder in a trussed configuration while the annuloplasty ring is delivered and secured to the implantation site. Once the ring is secured at the annulus, the truss suture is severed with one cut, enabling the hinged wings to pivot and the holder to be pulled free of the ring.

### RELATED APPLICATIONS

This application is a continuation of International Patent Application No. PCT/US2024/46565, filed September 13, 2024, which claims the benefit of U.S. Patent Application No. 63/582,809, filed September 14, 2023, the entire contents all of which are expressly incorporated by reference.

### TECHNICAL FIELD

The present disclosure relates generally to annuloplasty ring holders, and in particular to a holder that is easily assembled and detached from an annuloplasty ring.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Various surgical techniques may be used to repair a diseased or damaged valve. A commonly used repair technique effective in treating mitral valve incompetence is annuloplasty, which often involves reshaping or remodeling the annulus by attaching a prosthetic annuloplasty repair segment or ring thereto. The procedure is done with the heart stopped and the patient on cardiopulmonary bypass (“on pump”). For instance, the goal of a posterior mitral annulus repair is to bring the posterior mitral leaflet forward toward to the anterior leaflet to improve leaflet coaptation. Annuloplasty rings may be stiff, flexible or semi-rigid, and a “remodeling” annuloplasty ring typically has an inner core that is “generally rigid” or “semi-rigid” in that it will flex to a small extent but resist distortion when subjected to the stress imparted thereon by the mitral valve annulus of an operating human heart.

Surgically implanted annuloplasty rings are typically delivered through the resected left atrium above the mitral valve while the heart is stopped. The annuloplasty ring is held at the periphery of a template or holder by connecting sutures through the ring while the surgeon advances the assembly on the end of a handle to the annulus. The ring is then sutured to the annulus, and the handle and template disconnected and removed, sometimes in two stages due to the handle being in the way.

There remains a need for an annuloplasty ring holder that can more easily be assembled and detached from the annuloplasty ring.

### SUMMARY

Disclosed here is a holder for an annuloplasty ring that detaches with one cut. The annuloplasty ring mounts around a periphery of the holder without the use of connecting sutures. The holder has a proximal plate defining a lip on one side of the ring, and a distal retainer that defines a second lip on the other side, the two lips retaining the ring around the holder periphery. The distal retainer is formed by two hinged wings. A truss suture maintains the holder in a trussed configuration while the annuloplasty ring is delivered and secured to the implantation site. Once the ring is secured at the annulus, the truss suture is severed with one cut, enabling the hinged wings to pivot and the holder to be pulled free of the ring.

A first system for holding and delivering an annuloplasty ring comprises a holder having a proximal plate opposite a distal retainer. The distal retainer includes a pair of hinged wings that extend outward from each other and attach to inner hinges to enable movement toward and away from the proximal plate. A truss suture secured to the holder passes through aligned holes in the proximal plate and distal retainer. The truss suture maintains the holder in a trussed configuration with the wings hinged into contact with the proximal plate. The holder defines a peripheral groove in the trussed configuration sized to receive an annuloplasty ring therein, the peripheral groove being formed on a distal side by the wings. The truss suture spans across a cutting guide projecting proximally upward from the proximal plate and is secured to the holder such that severing the truss suture at the cutting guide enables conversion of the holder from the trussed configuration to a relaxed configuration wherein the wings are free to pivot away from proximal plate and release the ring from the peripheral groove.

In the first system, the annuloplasty ring may be configured for implant at a mitral annulus and has a generally rounded D-shaped periphery with a major axis perpendicular to a minor axis, and the peripheral groove at least partly conforms to the periphery of the annuloplasty ring. The wings may be hinged to pivot about axes parallel to the minor axis. There is preferably only a single truss suture and a single cutting guide. The truss suture may be tied off on both wings and threads several times through the aligned holes before spanning the cutting guide. In one embodiment, both the proximal plate and the wings define channels between at least two of the aligned holes into which the truss suture is recessed. The peripheral groove is preferably defined by opposing concave surfaces radially inside of outer lips formed in both the proximal plate and the wings. The concave surface on the proximal plate may extend around the entire holder, while the combined concave surfaces on the two wings only extends part way around the holder.

The first system may also include an annuloplasty ring for implant at a mitral annulus having a generally rounded D-shaped periphery with a major axis perpendicular to a minor axis, wherein the peripheral groove at least partly conforms to the periphery of the annuloplasty ring. The annuloplasty ring may be size-adjustable, and includes an internal adjustment tether extending around the periphery of the ring in an internal lumen.

A second system for holding and delivering an annuloplasty ring comprises an annuloplasty ring for implant at a mitral annulus having a generally rounded D-shaped periphery with a major axis perpendicular to a minor axis. A ring holder has a proximal plate opposite a distal retainer, wherein the distal retainer includes a pair of wings that extend outward from each other and attach to the proximal plate to enable movement toward and away from the proximal plate. A truss suture secured to the holder maintains the holder in a trussed configuration with the wings held in contact with the proximal plate. The holder defines a peripheral groove in the trussed configuration sized to receive the annuloplasty ring therein, and the peripheral groove is formed on a distal side by the wings. The truss suture spans across a cutting guide projecting proximally upward from the proximal plate and is secured to the holder such that severing the truss suture at the cutting guide enables conversion of the holder from the trussed configuration to a relaxed configuration wherein the wings are free to move away from proximal plate and release the ring from the peripheral groove.

In the second system, the wings may be hinged to a bulkhead fixed with respect to the proximal plate and thus pivot about axes parallel to the minor axis. There may be only a single truss suture and a single cutting guide. The truss suture is preferably tied off on both wings and threads several times through aligned holes in the proximal plate and distal retainer before spanning the cutting guide. Both the proximal plate and the wings may define channels between at least two of the aligned holes into which the truss suture is recessed. Preferably, the peripheral groove is defined by opposing concave surfaces radially inside of outer lips formed in both the proximal plate and the wings. The concave surface on the proximal plate may extend around the entire holder, while the combined concave surfaces on the two wings only extends part way around the holder.

In the second system, the annuloplasty ring may be size-adjustable, and includes an internal adjustment tether extending around the periphery of the ring in an internal lumen. The annuloplasty ring may be partly formed by an internal coiled body and is expandable to enable manual expansion when mounting the ring into the peripheral groove of the holder in the trussed configuration. The annuloplasty ring may further include an outer sewing margin formed by a peripheral band of suture-permeable material captured within a peripheral pocket of an outer fabric cover on the ring.

A further understanding of the nature and advantages will become apparent by reference to the remaining portions of the specification and drawings.

### BRIEF DESCRIPTION OF THE DRAWINGS

Features and advantages of the present disclosure will become appreciated as the same become better understood with reference to the specification, claims, and appended drawings wherein:

FIG. 1A is a top plan view of an annuloplasty ring mounted on a template or holder of the present application, and FIG. 1B is a bottom plan view thereof;

FIGS. 2A and 2B are perspective views of an exemplary annuloplasty ring holder of the present application in a trussed configuration without an annuloplasty ring mounted thereon;

FIGS. 3A-3C are top plan, front elevational, and bottom plan views of the annuloplasty ring holder of the present application in a relaxed configuration;

FIG. 4 is a partially cutaway perspective view of an annuloplasty ring that may be mounted on the exemplary holders described herein, and FIG. 4A is a radial sectional view through one side thereof;

FIG. 5A is a front elevational view of the holder of the present application in the trussed configuration above the annuloplasty ring of FIG. 4 in sectional view just prior to mounting the ring on the holder, and FIG. 5B shows the assembly after mounting the ring on the holder; and

FIG. 6A is a top plan view of the assembled holder and ring indicating a one-cut detachment step, and FIG. 6B is a front elevational view after the one-cut detachment step showing the holder in the released configuration being detached from the annuloplasty ring.

### DETAILED DESCRIPTION

The right ventricle and left ventricle are separated from the right atrium and left atrium, respectively, by the tricuspid valve and mitral valve; e.g., the atrioventricular valves. Though annuloplasty rings for correction of the mitral annulus is the primary focus of the present application, it should be understood that the annuloplasty rings and holders therefor described herein may equally be used to treat the tricuspid valve, and thus the claims should not be constrained to the mitral side unless expressly limited.

The term “axis” in reference to the illustrated annuloplasty rings, and other non-circular or non-planar rings, refers to a line generally through the centroid of the ring periphery when viewed in plan view. “Axial” or the direction of the “axis” can also be viewed as being parallel to the average direction of blood flow within the valve orifice and thus within the ring when implanted therein. Stated another way, an implanted mitral ring orients about a central flow axis aligned along an average direction of blood flow through the mitral annulus from the left atrium to the left ventricle. The plan views of the annuloplasty rings illustrated herein are as looking from the atrial side in the direction of blood flow. For the purpose of orientation, therefore, the atrial side of the ring is up and the ventricular side is down.

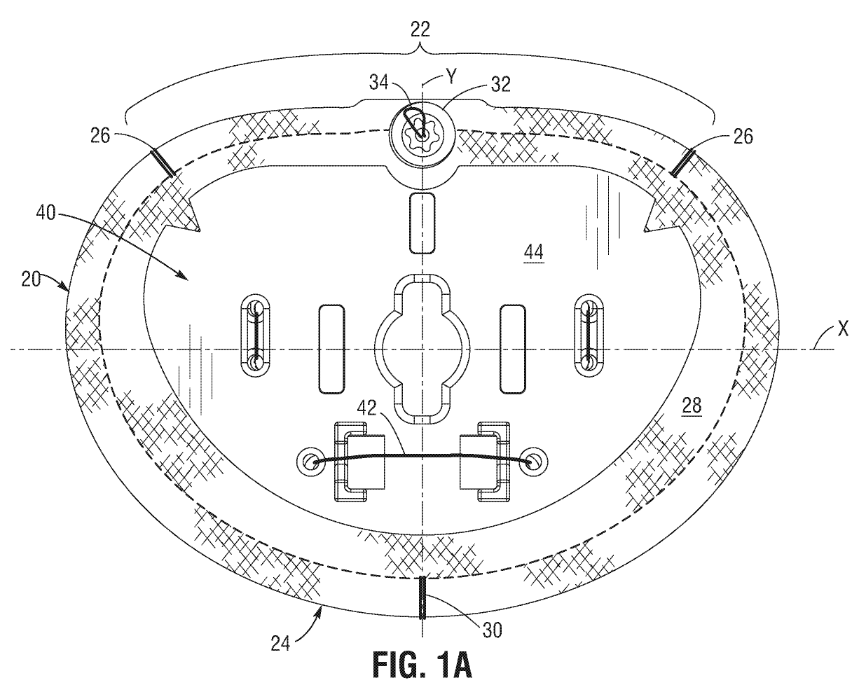

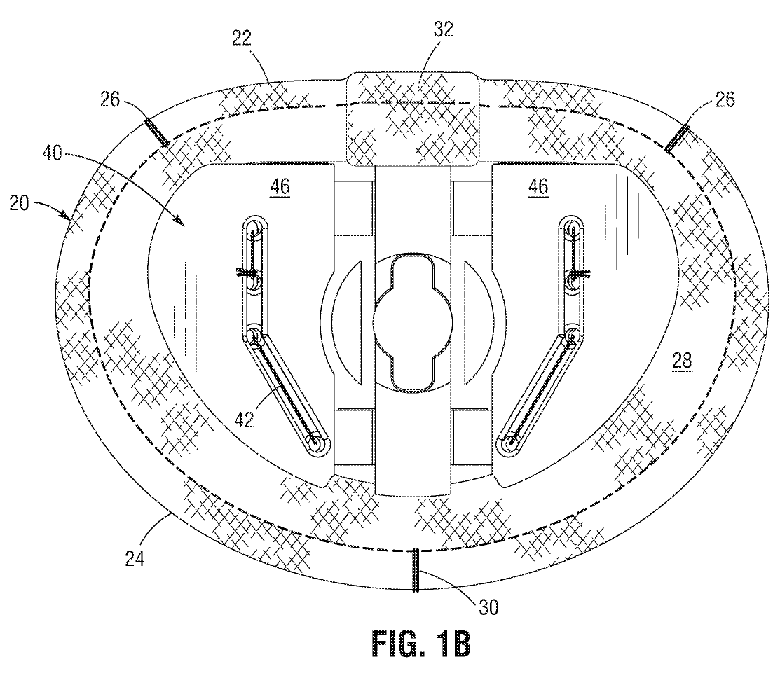

FIG. 1A is a top plan view of an annuloplasty ring 20 mounted on a template or holder 40 of the present application, and FIG. 1B is a bottom plan view thereof. The annuloplasty ring 20 is shown configured for repair of a mitral annulus, and as such has a generally rounded D-shape with a somewhat straight anterior peripheral aspect 22 opposite a more rounded posterior peripheral aspect 24. A major axis X across the annuloplasty ring 20 is indicated, as is a perpendicular minor axis Y. The anterior aspect 22 may be demarcated by a pair of trigone markers 26, typically provided by radially-oriented sutures sewn to an exterior fabric cover 28. The midpoint of the posterior aspect 24 may be indicated by a similar marker 30. As will be explained below, the annuloplasty ring 20 has a construction that enables its peripheral size to be adjusted, and as such has a junction housing 32 at the midpoint of the anterior aspect 22 with an adjustment tether 34 extending therefrom.

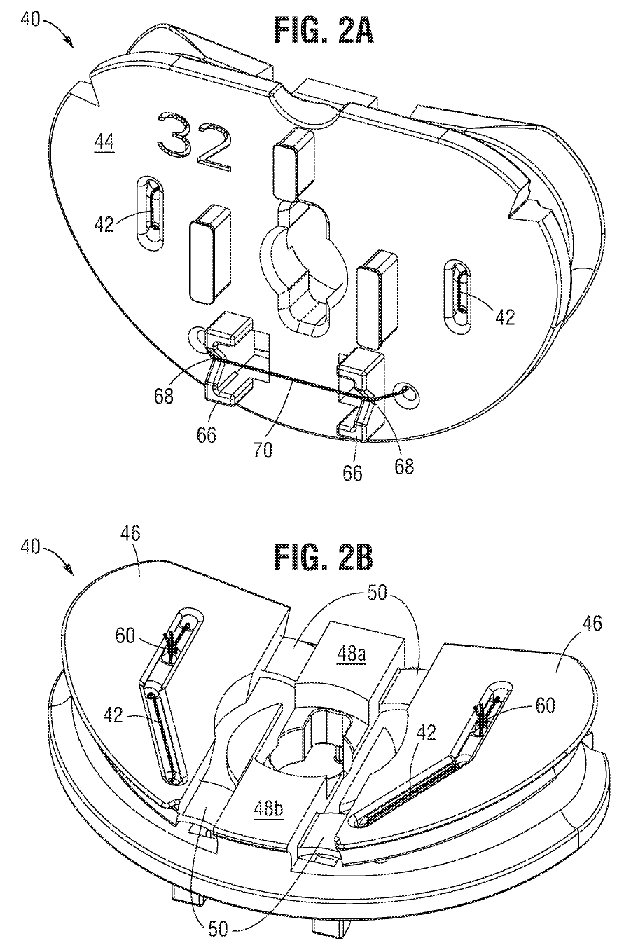

With reference also to the perspective views of FIGS. 2A and 2B, the exemplary annuloplasty ring holder 20 is shown in a trussed configuration with a single truss suture 42 incorporated therein. The holder 20 is composed of multiple movable parts, most prominently generally planar atrial or proximal plate 44 opposite a ventricular or distal retainer made up of multiple elements, including two hinged wings 46. More particularly, and with specific reference to FIG. 2B, aside from the hinged wings 46, the distal retainer comprises a central bulkhead defined by spaced apart bulkhead members 48a, 48b, and hinges 50. Each of the wings 46 is hingedly connected to both bulkhead members 48a, 48b via the hinges 50, as shown. The bulkhead members 48a, 48b are fixed to the distal side of the proximal plate 44, such as by being part of the same homogeneous part, and thus the wings 46 are hingedly connected to the underside of the proximal plate 44.

It should be understood that although the proximal plate 44 and the holder 40 in general are described and shown as being planar, they may also be three-dimensional. That is, some mitral annuloplasty rings are saddle-shaped to better conform to the typical contours of the mitral annulus, and the holder 40 may mimic that 3D shape so as to have an undulating peripheral groove to retain the ring. Also, FIG. 2A indicates a ring size “32” on the proximal plate 44. This pertains to a mm dimension of the major diameter X of the ring 20, which in this case is 32 mm, and helps ensure the right holder is used with a 32 mm ring while also providing quick identification of size of the holder.

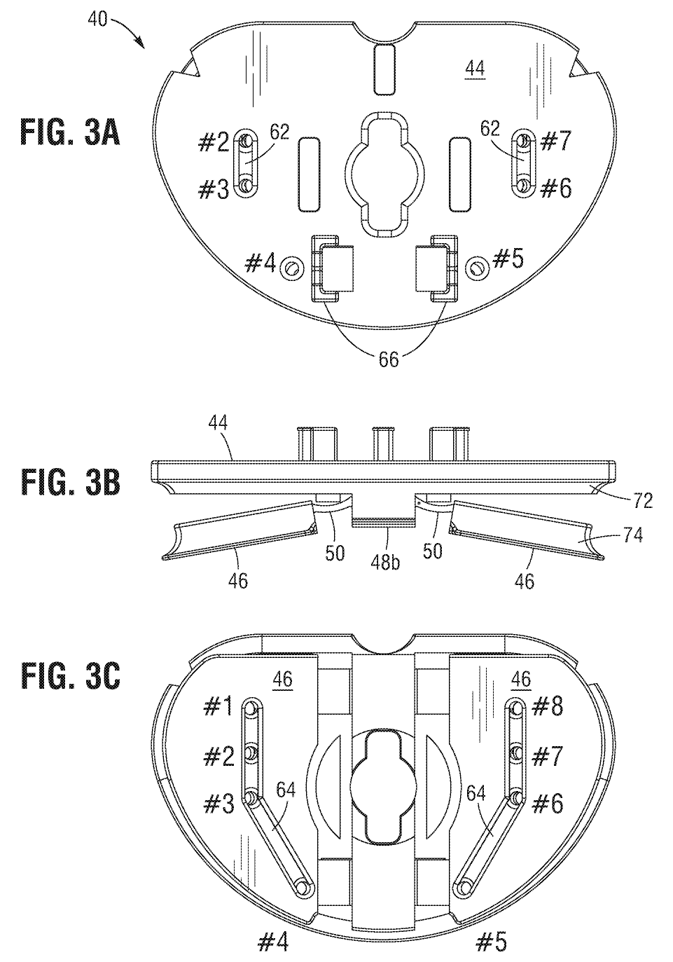

For the purpose of orientation, and relative to the annuloplasty ring 20 when mounted thereon, the bulkhead members 48a, 48b are aligned along the minor axis Y, with the wings 46 extending in opposite directions along the major axis X. Each of the wings 46 pivots relative to the bulkhead members 48a, 48b about the hinges 50 along axes which are generally parallel to the minor axis Y. The wings 46 pivot from a first position generally extending directly away from each other and parallel to the proximal plate 44 to a second position angled from the proximal plate 44. The wings 46 are shown in pivoted to the second position in FIG. 3B. These two positions will also be referred to as trussed and relaxed. Namely, when the truss suture 42 is threaded through the various parts of the holder 20 and pulled taut, the wings 46 are in the first or trussed position. Conversely, when the truss suture 42 has been severed or is not integrated with the holder 20, the wings 46 are in the second or relaxed position of FIG. 3B. The hinges 50 may be biased to urge the wings 46 into the relaxed position, or they may simply be relatively weak such that gravity causes the wings 46 to fall into the relaxed position when the holder 20 is oriented with the proximal plate 44 up, again as seen in FIG. 3B.

FIGS. 3A-3C show the annuloplasty ring holder 20 in the relaxed configuration. Both the proximal plate 44 and the wings 46 of the distal retainer have a number of through holes therein. Holes numbered #1–8 show on the distal retainer while only holes #3–8 show on the proximal plate 44. The truss suture 42 gets threaded back and forth through these holes and is secured taut to convert the holder 20 from the relaxed configuration to the trussed configuration.

Using a comparison between FIGS. 2A/2B and 3A/3C, one can see that the free ends of the truss suture 42 are secured using knots 60 to opposite wings 46 through holes #1–2 and #6–7. Starting from one of the knots 60 and hole #2 or hole #7, the truss suture 42 traverses across the proximal plate 44 between holes #2–3 and #7–6. A short channel 62 may be provided on the proximal plate 44 between holes #2–3 and #7–6, whereas on both distal retainer wings 46 an elongated angled channel 64 extends in sequence between holes #1–4 and #5–8. The truss suture 42 is thus recessed within the channels 62, 64 to help conceal the truss suture between holes and reduce any chance of snagging the suture during assembly or after.

From holes #3 and #6, the truss suture 42 then traverses to holes #4 and #5, respectively, passing within the angled channels 64 on the distal retainer wings 46 as seen in FIG. 3C. From the distal side, the truss suture 42 then passes up through holes #4 and #5 to the proximal plate 44 and spans across a cutting guide defined by two spaced-apart cutting guide brackets 66, as best seen in FIG. 2A. The cutting guide brackets 66 comprise walls projecting proximally up from the proximal plate 44 and defining central notches 68 which receive and center the truss suture 42. The spacing of the cutting guide brackets 66 exposes a mid-section length 70 of the truss suture 42 on the proximal plate 44 for eventual severing when needed. The cutting guide brackets 66 are located so that the mid-section length 70 is centered on and perpendicular to the minor axis Y, at a location about halfway between the center of the ring 20 and holder 40 and the posterior aspect 24 of the ring. This location is away from the junction housing 32 to which a holder (see below) attaches, and thus provides good visibility for the surgeon to sever the mid-section length 70 on the cutting guide.

Consequently, by tying the truss suture 42 to holes #1–2 (or #7–8), the assembler can thread the truss suture 42 around the holder 20 until it reaches holes #7–8 (or #1–2) where it is tied off with a knot 60. Prior to tying the truss suture 42 to holes #7–8 the assembler pulls the suture taut to maintain the holder 20 in its trussed configuration. This pulls the wings 26 up against an underside of the proximal plate 44.

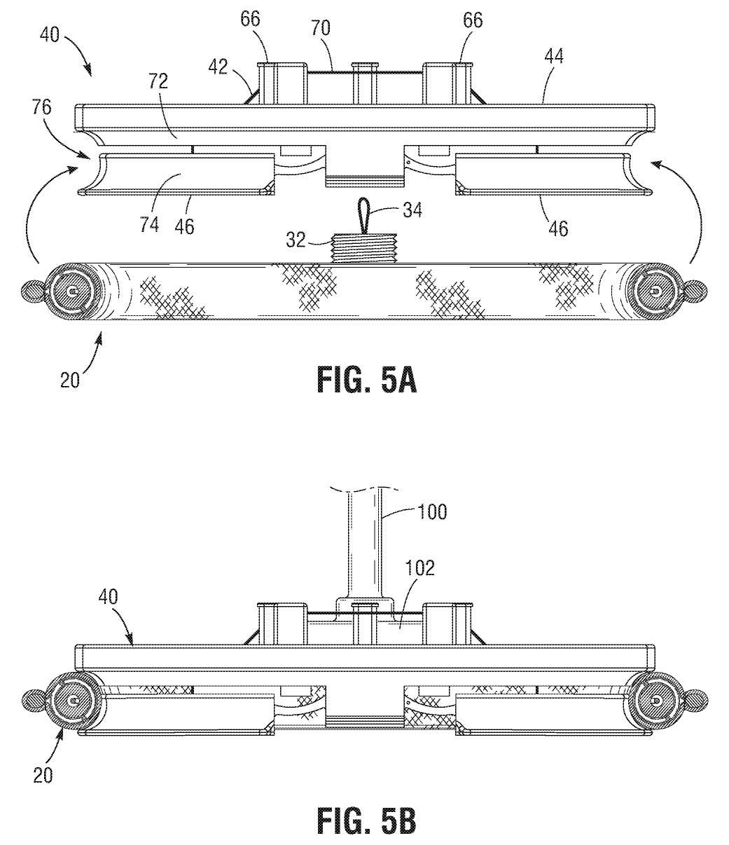

With reference back to FIGS. 3A-3C, a peripheral edge of the proximal plate 44 defines an outer lip leading to a concave surface 72 angled in a distal direction, while both wings 46 define an outer lip leading to mirror concave surfaces 74 partially around their outer peripheries. When the holder 20 is in the trussed configuration, the combination of the outer lips and opposed concave surfaces 72, 74 defines a generally semi-circular groove 76 for receiving and retaining the annuloplasty ring 20, as seen in FIG. 5A. Prior to an explanation of mounting and then detaching the ring 20 from the holder 40, a better understanding of the adjustable annuloplasty ring is in order.

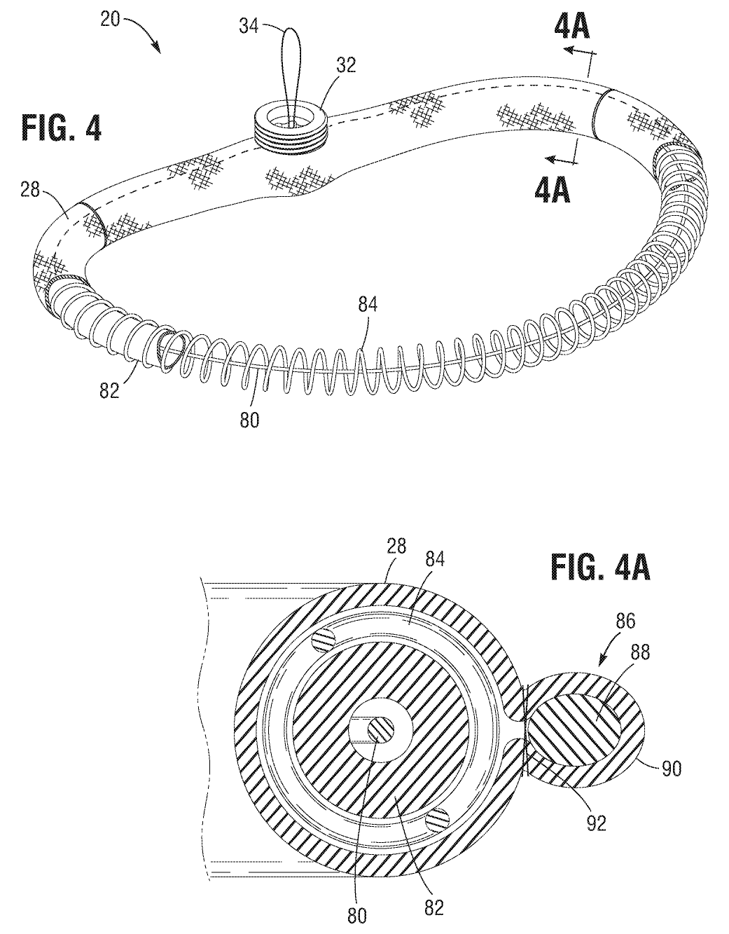

FIG. 4 is a partially cutaway perspective view of the annuloplasty ring 20, and FIG. 4A is a radial sectional view through one side thereof. The adjustment tether 34 has a continuous central loop 80 that passes through an inner lumen of the ring. As seen in the cross-section of FIG. 4A, the central loop 80 extends through an inner lumen of a compressible filler member 82 that in turn passes through a helically coiled body 84. Although not shown, the coiled body 84 is secured to tubular extensions of the junction housing 32.

The exposed portion of the adjustment tether 34 at the junction housing 32 may be pulled to cinch the ring 20 to a smaller overall shape. Both the coiled body 84 and filler member 82, not to mention the outer fabric cover 66, are flexible in terms of constriction, and thus the adjustment tether 34 determines their final size. The coiled body 84 is desirably a highly elastic metal such as nitinol, and may be heat set into the illustrated shape. The tubular filler member 82 may be silicone or other soft material such as fabric. The adjustment tether 34, on the other hand, is made of a metal such as a cobalt chromium alloy which resists creep. After tensioning the ring 20 to the proper size, the adjustment tether 34 can be locked in its reduced diameter constricted state within the junction housing 32. Due to the metallic nature of the adjustment tether 34 the ring size remains the same after constriction and is not subject to stretching from material creep.

Further details of the adjustable annuloplasty ring 20 are disclosed in International Patent Application No. PCT/US23/14440, filed March 23, 2023, the contents of which are expressly incorporated herein for all purposes.

FIG. 4A also shows an outer sewing margin 86 formed around the outer periphery of the ring 20. For example, the sewing margin 86 may be created by a peripheral band 88 of suture-permeable material such as silicone or fabric captured within a peripheral pocket 90 of the outer fabric cover 28. Stitches 92 may be used to hold the band 88 within the pocket 90, and thus maintain the shape of the sewing margin 86. The sewing margin 86 is extremely helpful when anchoring the annuloplasty ring 20 to the native annulus. That is, anchoring sutures are typically utilized to secure the ring 20 to the annulus, and providing an enlarged sewing margin 86 as shown makes the task much easier. Furthermore, the sewing margin 86 reduces the risk of looping any of the anchoring sutures through the coiled internal structure of the ring.

With reference now to FIG. 5A, assembly of the annuloplasty ring 20 with the holder 40 will be described. As mentioned above, the holder 40 in its trussed configuration defines a peripheral concave grooves 76. The annuloplasty ring 20 can be manually mounted around the holder 40 in the grooves 76 by simply expanding the ring, either by hand or using some sort of expander tool. Because of the flexible nature of the components of the ring 20, in particular the coiled body 84, it is relatively elastic. Of course, this presupposes that the adjustment tether 34 remains loose.

FIG. 5B shows the ring 20 mounted around the periphery of the holder 40 in its trussed configuration. A delivery handle 100 is shown attached to the junction housing 32 on the anterior aspect of the ring 20. The delivery handle 100 may terminate in a generally cup-shaped distal end 102 that couples to the junction housing 32, such as with threads as shown. The delivery handle 100 includes an internal adjustment mechanism (not shown) that engages the adjustment tether 34, enabling tensioning of the tether and reduction in size of the ring 20. Again, details of such a delivery handle may be seen in International Patent Application No. PCT/US23/14440. The delivery handle 100 may be sufficiently long to extend from outside the body into proximity of the target annulus so that the size of the ring 20 can be adjusted remotely.

Implantation of the annuloplasty ring 20 begins with establishing cardiopulmonary bypass on the patient and opening an access pathway to the target annulus, typically the mitral annulus. The annuloplasty ring 20 is then advanced on the holder 40 using the delivery handle 100 until it is seated against the annulus. At this stage, the delivery handle 100 may be detached from the holder 40 to provide greater access for tying off of the anchoring sutures through the sewing margin 86. Alternatively, the delivery handle 100 remains attached for subsequent size adjustment of the ring 20.

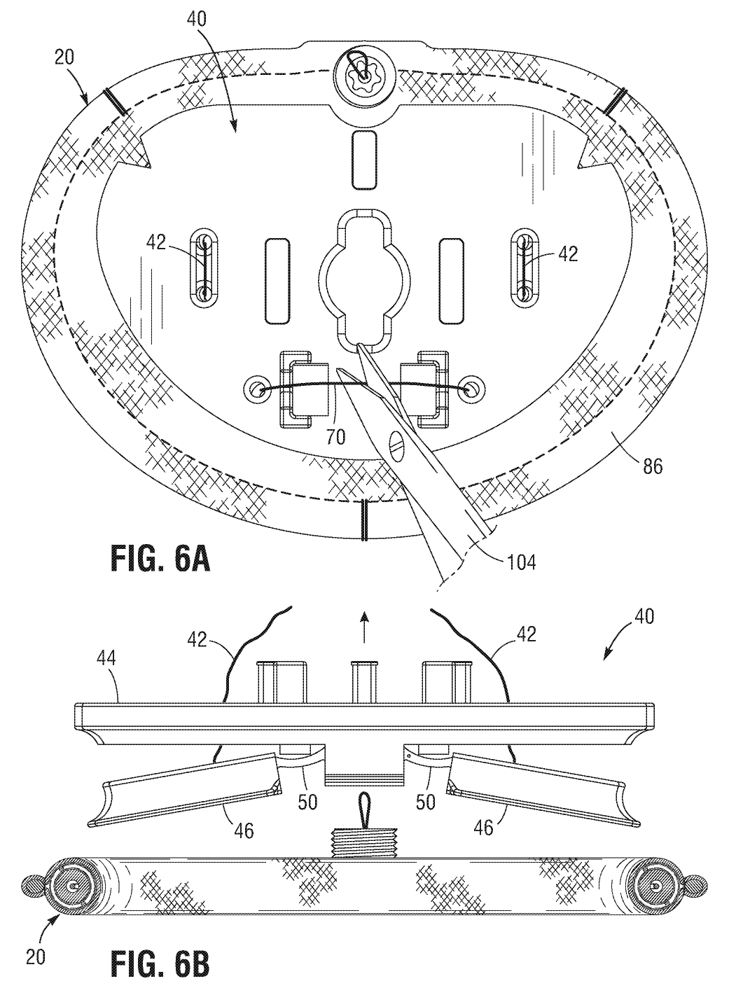

FIGS. 6A and 6B illustrate a one-cut detachment step showing the holder 40 in the released configuration being detached from the annuloplasty ring. A sharp instrument such as a scalpel or scissors 100 is introduced into the surgical field and the midsection 70 of the truss suture 42 is severed. This releases tension on the truss suture 42 to enable conversion of the holder 40 from the trussed configuration to the relaxed configuration. FIG. 6B shows the severed ends of the truss suture 42 and the holder 40 being lifted free of the annuloplasty ring 20. Because the distal wings 46 are now free to pivot about the hinges 50, the surgeon can simply pull the entire holder 40 upward away from the ring 20, which remains secured to the annulus.

FIG. 6B effectively illustrate to beneficial aspects of the present application. First of all, there are no connecting sutures passing through both ring 20 in the holder 40. Prior annuloplasty ring holders required threading of connecting sutures through the ring, which required them to be pulled free from within the ring when detaching the holder. The present holder 40 simply lists free from the ring, with the truss suture 42 being solely connected to the various parts of the holder and never passing through the ring 20. Secondly, the holder 40 can be released using one cut. Many prior annuloplasty ring holders required two or more steps to sever the connecting sutures that, again, pass through the ring. These beneficial aspects make for an elegant and efficient annuloplasty ring holder release.

While the foregoing is a complete description of the preferred examples, various alternatives, modifications, and equivalents may be used. Moreover, it will be obvious that certain other modifications may be practiced within the scope of the appended claims.

What is claimed is:

1. A system for holding and delivering an annuloplasty ring, comprising:
a holder having a proximal plate opposite a distal retainer, wherein the distal retainer includes a pair of hinged wings that extend outward from each other and attach to inner hinges to enable movement toward and away from the proximal plate; and
a truss suture secured to the holder and passed through aligned holes in the proximal plate and distal retainer, wherein the truss suture maintains the holder in a trussed configuration with the wings hinged into contact with the proximal plate, and wherein the holder defines a peripheral groove in the trussed configuration sized to receive an annuloplasty ring therein, the peripheral groove being formed on a distal side by the wings, the truss suture spanning across a cutting guide projecting proximally upward from the proximal plate and being secured to the holder such that severing the truss suture at the cutting guide enables conversion of the holder from the trussed configuration to a relaxed configuration wherein the wings are free to pivot away from proximal plate and release the ring from the peripheral groove.

2. The system of claim 1, wherein the annuloplasty ring is configured for implant at a mitral annulus and has a generally rounded D-shaped periphery with a major axis perpendicular to a minor axis, and the peripheral groove at least partly conforms to the periphery of the annuloplasty ring.

3. The system of claim 2, wherein the wings are hinged to pivot about axes parallel to the minor axis.

4. The system of claim 1, wherein there is a single truss suture and a single cutting guide.

5. The system of claim 1, wherein the truss suture is tied off on both wings and threads several times through the aligned holes before spanning the cutting guide.

6. The system of claim 5, wherein both the proximal plate and the wings define channels between at least two of the aligned holes into which the truss suture is recessed.

7. The system of claim 1, wherein the peripheral groove is defined by opposing concave surfaces radially inside of outer lips formed in both the proximal plate and the wings.

8. The system of claim 7, wherein the concave surface on the proximal plate extends around the entire holder, while the combined concave surfaces on the two wings only extends part way around the holder.

9. The system of claim 1, further including an annuloplasty ring for implant at a mitral annulus having a generally rounded D-shaped periphery with a major axis perpendicular to a minor axis, and the peripheral groove at least partly conforms to the periphery of the annuloplasty ring.

10. The system of claim 9, wherein the annuloplasty ring is size-adjustable, and includes an internal adjustment tether extending around the periphery of the ring in an internal lumen.

11. A system for holding and delivering an annuloplasty ring, comprising:
an annuloplasty ring for implant at a mitral annulus having a generally rounded D-shaped periphery with a major axis perpendicular to a minor axis;
a ring holder has a proximal plate opposite a distal retainer, wherein the distal retainer includes a pair of wings that extend outward from each other and attach to the proximal plate to enable movement toward and away from the proximal plate; and
a truss suture secured to the holder that maintains the holder in a trussed configuration with the wings held in contact with the proximal plate, wherein the holder defines a peripheral groove in the trussed configuration sized to receive the annuloplasty ring therein, the peripheral groove being formed on a distal side by the wings, the truss suture spanning across a cutting guide projecting proximally upward from the proximal plate and being secured to the holder such that severing the truss suture at the cutting guide enables conversion of the holder from the trussed configuration to a relaxed configuration wherein the wings are free to move away from proximal plate and release the ring from the peripheral groove.

12. The system of claim 11, wherein the wings are hinged to a bulkhead fixed with respect to the proximal plate and pivot about axes parallel to the minor axis.

13. The system of claim 11, wherein there is a single truss suture and a single cutting guide.

14. The system of claim 11, wherein the truss suture is tied off on both wings and threads several times through aligned holes in the proximal plate and distal retainer before spanning the cutting guide.

15. The system of claim 14, wherein both the proximal plate and the wings define channels between at least two of the aligned holes into which the truss suture is recessed.

16. The system of claim 11, wherein the peripheral groove is defined by opposing concave surfaces radially inside of outer lips formed in both the proximal plate and the wings.

17. The system of claim 16, wherein the concave surface on the proximal plate extends around the entire holder, while the combined concave surfaces on the two wings only extends part way around the holder.

18. The system of claim 11, wherein the annuloplasty ring is size-adjustable, and includes an internal adjustment tether extending around the periphery of the ring in an internal lumen.

19. The system of claim 18, wherein the annuloplasty ring is partly formed by an internal coiled body and is expandable to enable manual expansion when mounting the ring into the peripheral groove of the holder in the trussed configuration.

20. The system of claim 18, wherein the annuloplasty ring further includes an outer sewing margin formed by a peripheral band of suture-permeable material captured within a peripheral pocket of an outer fabric cover on the ring.
